(19) United States
(12) Patent Application Publication
(10) Pub. No.: US 2019/0083916 A1
(43) Pub. Date: Mar. 21, 2019

(54) Scent Releasing Filter Assembly

(71) Applicant: John DeOliveira, Mississauga (CA)

(72) Inventor: John DeOliveira, Mississauga (CA)

(21) Appl. No.: 15/710,930

(22) Filed: Sep. 21, 2017

(51) Int. Cl. B01D 46/00; B01D 46/54; A61L 9/12; F24F 3/16

(52) U.S. Cl. CPC B01D 46/0038; B01D 46/543; B01D 46/0005; A61L 9/12; F24F 2003/1689; B01D 2279/50; A61L 2209/14; A61L 2209/16; F24F 3/1603

(57) ABSTRACT

A scent releasing filter assembly for enhancing an odor of a building includes a filter that may be positioned in an air supply of an HVAC system to filter air that is urged through the air supply. A chemical scent is infused into the filter. The chemical scent is released into the air urged by the HVAC system thereby enhancing an odor of a building.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to filter devices and more particularly pertains to a new filter device for enhancing an odor of a building.

### BRIEF SUMMARY OF THE INVENTION

An embodiment of the disclosure meets the needs presented above by generally comprising a filter that may be positioned in an air supply of an HVAC system to filter air that is urged through the air supply. A chemical scent is infused into the filter. The chemical scent is released into the air urged by the HVAC system thereby enhancing an odor of a building.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a scent releasing filter assembly according to an embodiment of the disclosure.

FIG. 2 is a top view of an embodiment of the disclosure.

FIG. 3 is a cross sectional view taken along line 3-3 of FIG. 2 of an embodiment of the disclosure.

FIG. 4 is a detail view taken from circle 4 of FIG. 3 of an embodiment of the disclosure.

FIG. 5 is a perspective in-use view of an embodiment of the disclosure.

### DETAILED DESCRIPTION OF THE INVENTION

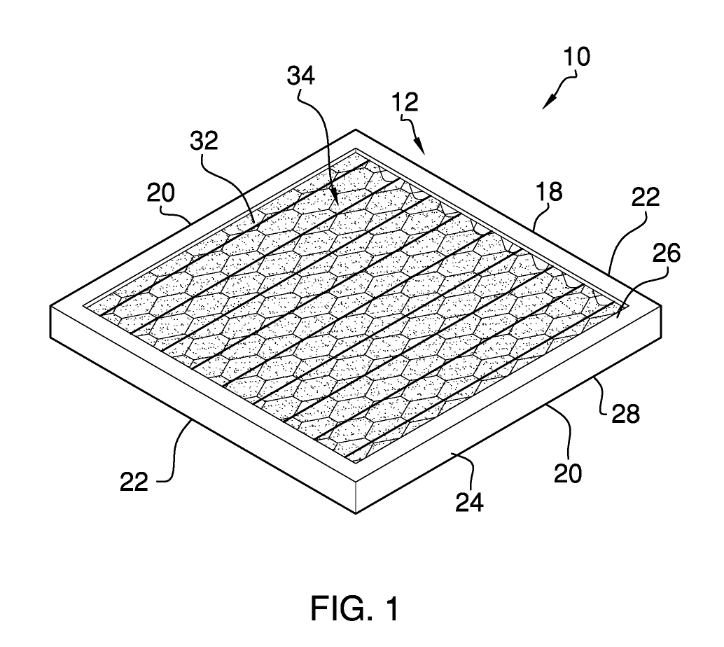

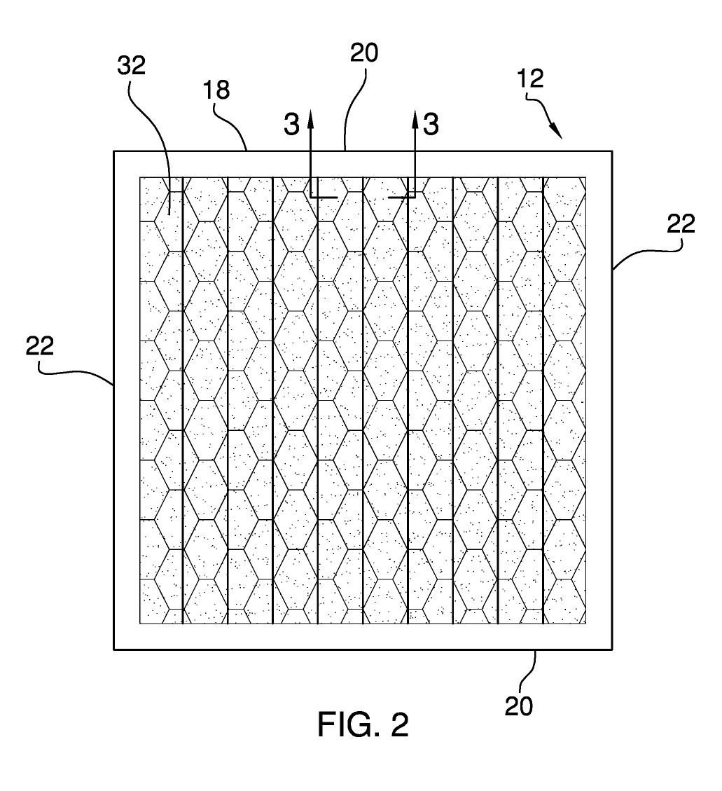

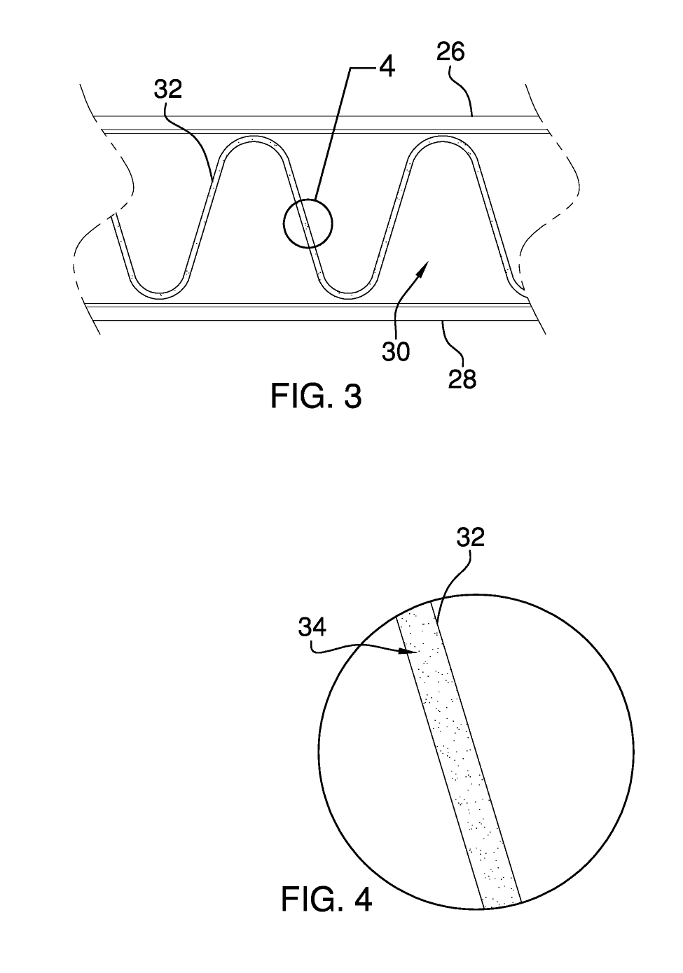

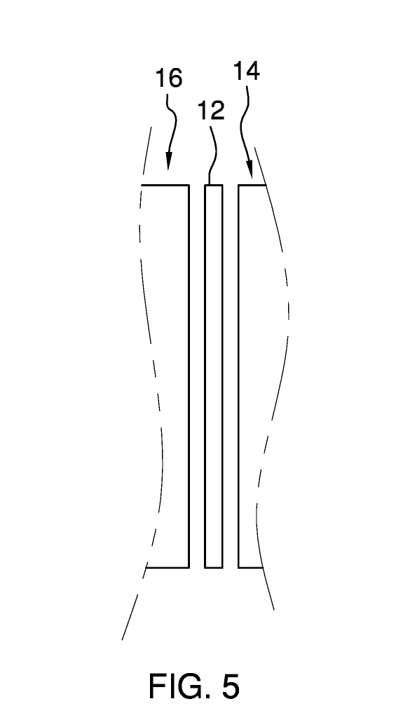

With reference now to the drawings, and in particular to FIGS. 1 through 5 thereof, a new filter device embodying the principles and concepts of an embodiment of the disclosure and generally designated by the reference numeral 10 will be described.

As best illustrated in FIGS. 1 through 5, the scent releasing filter assembly 10 generally comprises a filter 12 that is positioned in an air supply 14 of an HVAC system 16. The filter 12 filters air that is urged through the air supply 14. The HVAC system 16 may be a forced air system in a house or the like. The filter 12 may be an HVAC filter of any conventional design and dimension.

The filter 12 includes a frame 18 that has a pair of first sections 20 extending between a pair of second sections 22. The first sections 20 are spaced apart from each other such that the frame 18 forms a rectangular shape. Each of the first 20 and second 22 sections has a central panel 24 extending between a top panel 26 and a bottom panel 28. The top panel 26 and the bottom panel 28 corresponding to each of the first 20 and second 22 sections are spaced apart from each other to define a space 30 in the frame 18.

A membrane 32 is positioned in the frame 18 and the membrane 32 is comprised of a fluid permeable material to filter particles from the air urged through the air supply 14. The membrane 32 extends into the space 30 corresponding to each of the first 20 and second 22 sections of the frame 18. Moreover, the membrane 32 may be folded into an alternating sequence of pleats. The membrane 32 may be comprised of a material common to furnace filters.

A chemical scent 34 is provided and the chemical scent 34 is infused into the filter 12 wherein. In this way the chemical scent 34 releases a scent into the air urged by the HVAC system 16 thereby enhancing an odor of the building. The chemical scent 34 is infused into the membrane 32. Additionally, the chemical scent 34 may be essential oils or the like. The chemical scent 34 may have the scent of flowers, spices and any other pleasing scent. Additionally, the chemical scent 34 may have a service life of approximately thirty days.

In use, the filter 12 is positioned in the air supply 14 of the HVAC system 16 to filter air and to release the scent into the house. Moreover, the filter 12 is positioned in the HVAC system 16 when the house is on the market for selling. In this way the house has a pleasing aroma when potential buyers are touring the house thereby enhancing the potential buyer's impression of the house. Additionally, the filter 12 is out of sight when the filter 12 is positioned in the HVAC system 16 thereby eliminating unsightly air fresheners or the like in the house. The chemical scent 34 is exhausted after approximately thirty days. In this way a user is reminded to change the filter 12 when the scent is no longer detected in the house thereby ensuring that the filter 12 is regularly changed.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A scent releasing filter assembly being configured to be positioned in an HVAC system thereby facilitating a scent to be distributed through a building, said assembly comprising:
a filter being configured to be positioned in an air supply of an HVAC system wherein said filter is configured to filter air being urged through the air supply; and
a chemical scent being infused into said filter wherein said chemical scent is configured to release a scent into the air urged by the HVAC system thereby enhancing an odor of the building.

2. The assembly according to claim 1, wherein said filter comprises a frame having a pair of first sections extending between a pair of second sections, said first sections being spaced apart from each other such that said frame forms a rectangular shape.

3. The assembly according to claim 2, wherein each of said first and second sections has a central panel extending between a top panel and a bottom panel, said top panel and bottom panel being spaced apart from each other to define a space in said frame.

4. The assembly according to claim 2, further comprising a membrane being positioned in said frame, said membrane being comprised of a fluid permeable material wherein said membrane is configured to filter particles from the air urged through the air supply.

5. The assembly according to claim 4, wherein:
said frame has a pair of first sections and a pair of second sections, each of said first and second sections has a space therein; and
said membrane extending into said space corresponding to each of said first and second sections of said frame, said membrane being folded into an alternating sequence of pleats.

6. The assembly according to claim 5, wherein said chemical scent is infused into said membrane.

7. A scent releasing filter assembly being configured to be positioned in an HVAC system thereby facilitating a scent to be distributed through a building, said assembly comprising:
a filter being configured to be positioned in an air supply of an HVAC system wherein said filter is configured to filter air being urged through the air supply, said filter comprising:
a frame having a pair of first sections extending between a pair of second sections, said first sections being spaced apart from each other such that said frame forms a rectangular shape, each of said first and second sections having a central panel extending between a top panel and a bottom panel, said top panel and bottom panel being spaced apart from each other to define a space in said frame, and
a membrane being positioned in said frame, said membrane being comprised of a fluid permeable material wherein said membrane is configured to filter particles from the air urged through the air supply, said membrane extending into said space corresponding to each of said first and second sections of said frame, said membrane being folded into an alternating sequences of pleats; and
a chemical scent being infused into said filter wherein said chemical scent is configured to release a scent into the air urged by the HVAC system thereby enhancing an odor of the building, said chemical scent being infused into said membrane.
